(19) United States
(12) Patent Application Publication
(10) Pub. No.: US 2011/0105903 A1
(43) Pub. Date: May 5, 2011

(54) ORGANIC PIEZOELECTRIC MATERIAL, ULTRASONIC VIBRATOR, AND ULTRASONIC IMAGE DIAGNOSIS APPARATUS FOR MEDICAL APPLICATION

(76) Inventor: Kenji Ohnuma, Tokyo (JP)

(21) Appl. No.: 13/000,358

(22) PCT Filed: Feb. 25, 2009
(86) PCT No.: PCT/JP2009/053373

§ 371 Date: Dec. 20, 2010

(30) Foreign Application Priority Data
Jul. 3, 2008 (JP) 2008-174577

(51) Int. Cl. A61B 8/14; C08F 214/22

(52) U.S. Cl. 600/443; 526/255

(57) ABSTRACT

This invention provides an organic piezoelectric material for constituting an ultrasonic vibrator, which has excellent piezoelectric properties and is suitable in high-frequency and broad bands, an ultrasonic probe using the organic piezoelectric material, and an ultrasonic image diagnosis apparatus for medical application. The organic piezoelectric material is a film-shaped organic piezoelectric material and is characterized in that the organic piezoelectric material is produced by a process comprising performing heat treatment at a temperature at or above room temperature and at or below a temperature of 10° C. below the melting point of the organic piezoelectric material while applying tension and subsequently performing relaxation treatment in a period between the completion of the heat treatment and the time when the material has been cooled to room temperature.

### TECHNICAL FIELD

The present invention relates to an organic piezoelectric material for constituting an ultrasonic transducer suitable for a high frequency and a wide band, an ultrasonic transducer employing it, and an ultrasonic medical image diagnostic apparatus.

### BACKGROUND ART

Generically, sound waves of 16 kHz or more are collectively called ultrasonic waves, and since ultrasonic waves makes it possible to investigate inside nondestructively and harmless, ultrasonic waves are applied to various fields, such as an examination of defects and diagnosis of diseases. One of the various fields is an ultrasonic diagnostic apparatus which scans the inside of an analyte with ultrasonic waves and creates an image of the internal state of the analyte on a basis of reception signals generated from reflected waves (echo) of the ultrasonic waves from the analyte. This ultrasonic diagnostic apparatus employs an ultrasound probe which transmits and receives ultrasonic waves for an analyte. Such an ultrasound probe incorporates an ultrasonic wave transmission and reception element including a transducer which causes mechanical vibration based on transmission signals so as to generate ultrasonic waves and receives reflected wave of ultrasonic waves generated by difference in acoustic impedance at the inside of an analyte so as to generate reception signals.

In recent years, studied and developed is a harmonic imaging technique which forms an image of an internal state in an analyte not by a frequency (fundamental frequency) component of ultrasonic waves transmitted from an ultrasound probe into an analyte, but by its harmonic frequency component. This harmonic imaging technique has the following various advantages; (1) a side lobe level is small as compared with the level of a fundamental frequency component, a S/N ratio (signal to noise ratio) becomes good, whereby contrast resolution is improved, (2) since a frequency becomes high, a beam width becomes thin, whereby a cross direction resolution is improved, (3) since, in a close range, sound pressure is small and there is little fluctuation of sound pressure, multiple reflection is suppressed, and (4) decay at a position distant from a focal point is the same level as a fundamental wave, a bottom speed is made large as compared with the case where a high frequency wave is used as a fundamental wave.

Am ultrasound probe for this harmonic imaging is required to have a wide frequency band from the frequency of a fundamental wave to the frequency of a harmonic, and a frequency region at a low frequency side is used for transmission to transmit a fundamental wave. On the other hand, a frequency region at a high frequency side is used for reception to receive a harmonic wave (for example, refer to Patent Document 1).

The ultrasound probe disclosed in Patent Document 1 is an ultrasound probe which is brought in contact with an analyte so as to transmit ultrasonic waves to the inside of the analyte and to receive returned ultrasonic waves having reflected on the inside of the analyte. This ultrasound probe is provided with a first piezoelectric layer which is composed of a plurality of arranged first piezoelectric elements having predetermined first acoustic impedance and conducts transmission to transmit a fundamental wave composed of an ultrasonic wave with a prescribed central frequency into a analyte and reception to receive the fundamental wave among returned ultrasonic waves having reflected on the inside of the analyte. Further, the above ultrasound probe is provided with a second piezoelectric layer which is composed of a plurality of arranged second piezoelectric elements having predetermined second acoustic impedance smaller than the above first acoustic impedance and conducts reception to receive harmonic waves among returned ultrasonic waves having reflected on the inside of the analyte. The second piezoelectric layer is superimposed on the entire surface of the first piezoelectric layer at the side where the ultrasound probe is brought in contact with the analyte. With this structure, the ultrasound probe can conducts transmission and reception of ultrasonic waves in such a wide frequency band.

It is preferable that the fundamental wave in harmonic imaging is a sound wave whose band width is narrower as far as possible. As a piezoelectric element for such a sound wave, widely utilized is an inorganic piezoelectric body so called ceramics in which a rock crystal, a single crystal of LiNbO₃, LiTaO₃, or KNbO₃, a thin film of ZnO or AlN, or a sintered body of a Pb(Zr, Ti)O₃ type is subjected to a polarization treatment. Since a piezoelectric element to detect reception waves at a high frequency side is required to have a sensitivity for a more wide band width, the above inorganic piezoelectric body is not suitable for this piezoelectric element. As a piezoelectric element suitable for a high frequency and a wide band width, well known is an organic piezoelectric body utilizing an organic polymer material such as polyvinylidene fluoride (hereafter, abbreviated “PVDF”) (for example, refer to Patent Documents 2). As compared with an inorganic piezoelectric body, this organic piezoelectric body has characteristics, large flexibility, easiness in being made to a thin film, a large area and a long size, and capability for being shaped in an arbitrary form or configuration.

However, as compared with an element made of an inorganic piezoelectric body, an element made of an organic piezoelectric body is not said to have a sufficient piezoelectric property. Accordingly, in order to raise an orientation property of molecule and an amount of polarization, well known is the application of additional treatment such as stretching of a film, a heat treatment at a temperature of a melting point or less and a polarizing method combining them (for example, refer to Patent documents 2, 3). However, if a piezoelectric body containing PVDF as a principal component is produced by the above well-known method, the piezoelectric properties are improved surely. However, since a degree of crystallinity is high, flexibility which is an advantage as an organic piezoelectric body is not only lost, but the piezoelectric body becomes brittle. Further, since PVDF has glass transition temperature lower than a room temperature, even if the PVDF is cooled down from a heat treatment temperature to a room temperature, molecular movement is not fully frozen. As a result, a film deforms in accordance with residual stress hiding in its inside and loses flatness remarkably. Namely, its processing adequacy for a probe used for an ultrasonic diagnostic apparatus becomes insufficient. Further, new problems peculiar to an ultrasound probe are caused, for example, the reception sensitivity of an ultrasound probe becomes lower, or electrical breakdown strength becomes lower.

Patent Document 1: Japanese Unexamined Patent Publication No. 11-276478, official report

Patent Document 2: Japanese Unexamined Patent Publication No. 60-217674, official report

Patent Document 1: Japanese Unexamined Patent Publication No. 4-69827, official report

### DISCLOSURE OF THE INVENTION

### Problems to be Solved by the Invention

The present invention has been made in view of the above problems and situations, and the problems to be solved are to provide an organic piezoelectric material for constituting a ultrasonic transducer which is excellent in piezoelectric properties and is suitable for a high frequency and wide band width, an ultrasound probe employing it, and an ultrasonic medical image diagnostic apparatus.

### Means for Solving the Problems

The above object of the present invention can be attained by the following structures

1. In an film-shaped organic piezoelectric material, the organic piezoelectric material is characterized in that the organic piezoelectric material is produced by being subjected to a heat treatment at a temperature from a room temperature or more to a temperature lower by 10° C. than a melting point of the organic piezoelectric material while being applied with tension, subsequently, by being subjected to relaxation treatment while being cooled to a room temperature.
2. The organic piezoelectric material described in the 1 is characterized in that the organic piezoelectric material is subjected to biaxially stretching or unaxially stretching, after the stretching, a stress applied on the organic piezoelectric material is not allowed to become zero, the organic piezoelectric material is produced by being subjected to a heat treatment at a temperature from a room temperature or more to a temperature lower by 10° C. than a melting point of the organic piezoelectric material while being applied with tension, successively, by being subjected to relaxation treatment while being cooled to a room temperature.
3. The organic piezoelectric material described in the 1 or 2 is characterized in that the heat treatment is conducted on a condition of a temperature of 100° C. or more and 140° C. or less for 30 minutes or more and 10 hours or less while the organic piezoelectric material is applied with tension, successively, the relaxation treatment is conducted by −15% or more and +10% or less in a direction in which the tension was applied while the organic piezoelectric material is cooled to a room temperature.
4. The organic piezoelectric material described in any one of the 1 to 3 is characterized in that the organic piezoelectric material is composed of a copolymer of vinylidene fluoride and trifluoro ethylene, and the copolymer contains the vinylidene fluoride in an amount of 95 to 60 mol % and the trifluoro ethylene in an amount of 5-40 mol %.
5. The organic piezoelectric material described in any one of the 2 to 4 is characterized in that the organic piezoelectric material has a electromechanical coupling factor of 0.3 or more.
6. In an ultrasonic transducer employing the organic piezoelectric material described in any one of the 1 to 5, the ultrasonic transducer is characterized in that the organic piezoelectric material is produced in such a way that the organic piezoelectric material is subjected to the relaxation treatment in a direction parallel to a long side direction of the ultrasonic transducer.
7. In an ultrasonic medical image diagnostic apparatus which comprises means for generating electric signals; an ultrasound probe in which a plurality of transducers to transmit ultrasonic waves to an analyte in response to the electric signals and to generate reception signals corresponding to refection waves received from the analyte are arranged; and image processing means for producing an image of the analyte corresponding to the reception signals produced by the ultrasound probe; the ultrasonic medical image diagnostic apparatus is characterized in that the ultrasound probe is provided with both of a ultrasonic transducer for transmission and a ultrasonic transducer for reception, and one or both of the ultrasonic transducers are the ultrasonic transducer described in the 6.

### EFFECT OF INVENTION

With the above means of the present invention, it becomes possible to provide an organic piezoelectric material for constituting a ultrasonic transducer which is excellent in piezoelectric properties and is suitable for a high frequency and wide band width, an ultrasound probe employing it and an ultrasonic medical image diagnostic apparatus.

### BEST MODE FOR CARRYING OUT TILE INVENTION

The present invention will be explained in more detail.

An ultrasonic receiving transducer of the present invention is an ultrasonic transducer which has an ultrasonic piezoelectric material and is used for a probe for an ultrasonic medical image diagnostic apparatus. The ultrasonic piezoelectric material is an organic piezoelectric material which contains vinylidene fluoride as a principal component, and the organic piezoelectric material is subjected to a heat treatment at a temperature of from a room temperature to a temperature lower by 10° C. than a melting point while being applied with tension, and successively the organic piezoelectric material is subjected to a relaxation treatment while being cooled to a room temperature. Preferably, the organic piezoelectric material has been stretched biaxially or uniaxially, and without allowing the stress applied on the organic piezoelectric material to become zero after the stretching, the organic piezoelectric material is subjected to a heat treatment at a temperature of (a melting point −10° C.) or less while being applied with tension, and successively subjected to a relaxation treatment while being cooled to a room temperature. More preferably, the organic piezoelectric material is subjected to a heat treatment at a temperature of 100° C. or more and 140° C. or less for a period of 30 minutes or more and 10 hours or less while being applied with tension, and is subjected to a relaxation treatment of −15% or more and +10% or less in the direction in which the tension has been applied while being cooled to a room temperature.

The ultrasonic transducer of the present invention can constitute an ultrasound probe in combination with other ultrasonic transducers. In this case, the ultrasound probe may be constituted by a combination of the ultrasonic transducer of the present invention and the same kind organic piezoelectric material or another known piezoelectric material, and the piezoelectric material may be an inorganic material or a polymer material, and further the combined material may be another polymer material being not a piezoelectric material. The ultrasound probe is a laminated transducer of two or more layers in which the above-mentioned materials are stacked or pasted together, and preferable is an embodiment that the thickness of the laminated transducer is 20 to 600 μm.

A production method of the ultrasonic transducer of the present invention is preferably a production method with an embodiment that a polarization treatment is conducted before the formation of electrodes provided on both surface of an organic piezoelectric material after the formation of an electrode only at one side or after the formation of electrodes at both sides. The polarization treatment is preferably a voltage applying treatment.

The ultrasonic transducer of the present invention can constitute an ultrasound probe by being combined with other ultrasonic transducers. In this case, preferable is an embodiment that the ultrasound probe comprises the ultrasonic transducer of the present invention and is a laminated transducer of two or more layers constituted such that the ultrasonic transducer is pasted on another polymer material different from the organic piezoelectric material constituting the ultrasonic transducer, and the thickness of the laminated transducer is 40 to 150 μm.

The ultrasonic transducer of the present invention or an ultrasound probe employing it may be used preferably for an ultrasonic medical image diagnostic apparatus.

Hereafter, the present invention and its structural elements, and the best embodiment and mode for carrying out the present invention will be explained in detail.

### (Ultrasonic Transducer)

The ultrasonic transducer of the present invention is employed for a probe (probe) for an ultrasonic medical image diagnostic apparatus provided with an ultrasonic transmitting transducer and an ultrasonic receiving transducer.

Generally, an ultrasonic transducer is constituted such that a pair of electrodes are arranged so as to sandwich a layer (or film) (hereafter, referred to as “a piezo electric body layer” or “piezoelectric body film”) composed of a film-shape piezoelectric material, and plural transducers are arranged, for example, in one dimension so as to constitute an ultrasound probe.

The predetermined number of plural transducers arranged in the long axis direction is set as a caliber, and the plural transducers belonging in the caliber have a function that the plural transducers are driven to emit an ultrasonic beam so as to converge the ultrasonic beam onto a measurement section in an analyte, and a function that the plural transducers receive a reflective echo of ultrasonic emitted from the analyte and convert the reflective echo into electric signals.

### (Organic Piezoelectric Material)

As an organic piezoelectric material as a constitution material of the piezoelectric material which constitutes the ultrasonic transducer of the present invention, any organic piezoelectric material may be employable regardless of a low molecule material and a high molecule (polymer) material. Examples of an organic piezoelectric material with low molecule include a phthalic ester type compound, a sulfenamide type compound, an organic compound having a phenol skeleton, and the like. Examples of an organic piezoelectric material with high molecule include polyvinylidene fluoride, a polyvinylidene fluoride type copolymer, polyvinylidene cyanide, vinylidene cyanide type copolymerization, odd number nylons, such as nylon 9 and nylon 11, aromatic nylon, alicyclic nylon, polylactic acid, polyhydroxy carboxylic acid, such as polyhydroxybutyrate, a cellulose type derivative, poly urea, and the like. From viewpoints of good piezoelectric properties, processability, easy availability and the like, the organic piezoelectric material is required to be an organic piezoelectric material with high molecule, especially, a polymer material containing vinylidene fluoride as a principal component.

Concretely, the organic piezoelectric material is required to be a homopolymer of polyvinylidene fluoride including a CF₂ group having a large dipole moment or a copolymer containing vinylidene fluoride as a principal component. As the second component in the copolymer, tetrafluoroethylene, a trifluoro ethylene, hexafluoropropane, chlorofluoroethylene, and the like may be employed.

For example, in the case of vinylidene fluoride/trifluoro ethylene copolymer, an electromechanical coupling factor (piezoelectricity effect) in the thickness direction changes depending on a copolymerization ratio. Therefore, the copolymerization ratio of the former is preferably 60 to 99 mol %, and more preferably 85 to 99 mol %.

A polymer which contains vinylidene fluoride in an amount of 85 to 99 mol % and perfluoroalkyl vinyl ether, perfluoroalkoxy ethylene, or perfluorohexa ethylene in an amount of 1 to 15 mol %, can raise the sensibility of a harmonic reception by suppressing a transmitted fundamental wave in a combination of an inorganic piezoelectric element for transmission and an organic piezoelectric element for reception.

As compared with an inorganic piezoelectric material composed of ceramics, the abovementioned organic piezoelectric material can be made into a thinned film. Therefore, the organic piezoelectric material is characterized in a point that it can be made into a transducer corresponding to the transmission and reception of high frequency wave.

In the present invention, the organic piezoelectric material is characterized to have a specific permittivity of 10 to 50 at a thickness resonance frequency. However, the specific permittivity can be adjusted by a quantity, composition, and a degree of polymerization of polar functional groups such as a CF₂ group or a CN group contained in a compound constituting the organic piezoelectric material and by a polarization treatment mentioned later.

The organic piezoelectric material constituting the transducer of the present invention may also be structured such that which multiple polymeric materials are laminated. In this case, as polymer materials to be laminated, in addition to the abovementioned polymer materials, the following polymer materials having relatively low specific permittivity can be employed in combination.

In the following exemplification, the numerical value in brackets represents the permittivity of a polymer material (resin). Examples of polymer materials include a methyl methacrylate resin (3.0), an acrylic nitrile resin (4.0), an acetate resin (3.4), an aniline resin (3.5), an aniline formaldehyde resin (4.0), an amino alkyl resin (4.0), an alkyd resin (5.0), nylon 6-6 (3.4), an ethylene resin (2.2), an epoxy resin (2.5), a vinyl chloride resin (3.3), a vinylidene chloride resin (3.0), a urea formaldehyde resin (7.0), a polyacetal resin (3.6), polyurethane (5.0), a polyester resin (2.8), polyethylene (low pressure) (2.3), polyethylene terephthalate (2.9), a polycarbonate resin (2.9), a melamine resin (5.1), a melamine formaldehyde resin (8.0), cellulose acetate (3.2), a vinyl acetate resin (2.7), a styrene resin (2.3), a styrene-butadiene rubber (3.0), a styrol resin (2.4), an ethylene fluoride resin (2.0), and the like.

The abovementioned polymer materials with the low specific permittivity may be selected appropriately in accordance with various objects such as adjustment of piezoelectric properties, provision of physical strength to an organic piezoelectric material and the like.

### (Producing Method of an Organic Piezoelectric Body Material)

An organic piezoelectric body material relating to the present invention contains the above polymer material as a principal structure component and can be produced by being subjected to a heat treatment at a temperature of from a room temperature to a temperature lower by 10° C. than a melting point while being applied with tension and successively being subjected to a relaxation treatment while being cooled to a room temperature.

In the case where an organic piezoelectric material containing the vinylidene fluoride relating to the present invention is made into an transducer, the organic piezoelectric material is shaped in the form of a film and a surface electrode to input electric signals is formed on it.

For forming a film, general methods, such as a melting method and a casting method, can be employed. In the case of a polyvinylidene fluoride-trifluoro ethylene copolymer, it is well know that this copolymer has a crystal form having spontaneous polarization only by being shaped in the form of a film. However, in order to improve the characteristics more, it is useful to subject such a copolymer to treatment to align the molecular arrangement.

As a stretching film production, various well-known methods are employable. For example, the above polymer material is dissolved in an organic solvent, such as ethyl methyl ketone (MEK), the resultant solution is cast on a support, such as a glass plate, and the casting layer is dried at normal temperature so as to obtain a film with a desired thickness. Further, the obtained film is stretched to a length with a predetermined magnification at a room temperature. In this stretching, the film is stretched uniaxially•biaxially to an extent that the organic piezoelectric material with a predetermined form is not destroyed. The stretching magnification is 2 to 10 times, and preferably 2 to 6 times.

In a vinylidene fluoride-trifluoro ethylene copolymer and/or a vinylidene fluoride-tetrafluoroethylene copolymer, a melt flow rate at 230° C. is 0.03 g/min or less. The melt flow rate is more preferably 0.02 g/min or less, and still more preferably 0.01 g/min or less. If a polymer piezoelectric body having such a melt flow, a thin film composed of a piezoelectric body with high sensitivity can be obtained.

Generally, in the case where a film-shaped material is subjected to a heat treatment, in order to provide heat efficiently and uniformly onto a film surface, it is desirable to place the film surface under temperature near a prescribed temperature while supporting an edge of the film with a chuck, a clip, and the like. At this time, in the case where a material of the film contracts by being heated, it is not desirable to provide heat in such a way that the film surface is brought in direct contact with a heat source such as a heat plate, because the flatness of the film may be spoiled. Rather, for the contract of the material by being heated, it is effective for the flatness to conduct a relaxation treatment. Herein, the relaxation treatment is conducted in such a way that during a heat treatment or in a process of cooling a film to a room temperature after the heat treatment, a stress on both ends of the film is changed while following a contracting or expanding force applied on the film. As long as a film can keep its flatness if the film slacks or as long as a film does not fracture if the stress applied on the film becomes large, the film may be shrunk so as to relax the stress or the film may be expanded so as to apply tension to an extent not to stretch. With regard to an amount of the relaxation treatment in the present invention, if a direction to stretch is defined as plus “+”, a negative relaxation treatment is conducted about 10% in length, and in the case where a film is extended while being cooled, it is conducted about 15%. There is fear that the relaxation treatment more than the above becomes stretching during cooling and causes film fracture.

As a heat treatment method of an organic piezoelectric material of the present invention, in order to provide heat efficiently and uniformly onto a film surface, it is desirable to place the film surface under temperature near a temperature whose upper limit is a temperature lower by 10° C. than a melting point of the film, while supporting an edge of the film with a chuck, a clip, and the like. In the case of an organic piezoelectric material containing polyvinylidene fluoride as a principal component, a melting point is in a range of 150° C. to 180 ° C. Therefore, it is desirable to conduct a heat treatment at 100° C. or more and 140° C. or less. With regard to the time of a heat treatment, a heat treatment conducted for 30 minutes or more exhibits its effect, and as the time of a heat treatment becomes longer, the growth of a crystal is advanced more. However, since the growth of a crystal saturates over time, actually, the time of a heat treatment may be about 10 hours, and about one whole day at longest.

### (Polarization Treatment)

As polarization treatment methods in the polarization treatment relating to the present invention, the conventionally well-known methods, such as a direct current voltage applying treatment, an alternating voltage applying treatment and a corona discharge treatment may be applied.

For example, in the case of the corona discharge treatment method, the corona discharge treatment may be conducted by an apparatus comprising a commercial high voltage power and electrode.

Since discharging conditions may differ depending on a device or a treatment environment, it is desirable to select conditions suitably. It may be preferable that the voltage of a high voltage power source is −1 to −20 kV, an electric current is 1 to 80 mA, the distance between electrodes is 1 to 10 cm, and an applied voltage is 0.5 to 2.0 MV/m.

The electrode may be preferably a needlelike electrode, a line electrode (wire electrode), and netlike electrode which are used conventionally. However, the present invention is not limited to them.

### (Substrate)

As a substrate, the selection of a substrate may differ depending on an intended us, a using method, and the like of an organic piezoelectric material relating to the present invention. In the present invention, examples of the substrate include a plastic plate or film of polyimide, polyamide, polyimidoamide, polyethylene terephthalate (PET), polyethylene enaphthalate (PEN), polymethyl methacrylate (PMMA), polycarbonate resin, and cycloolefin polymer. Further, the surface of these substrate materials may be covered with aluminium, gold, copper, magnesium, silicon, and the like. Furthermore, the substrate may be a plate or film of aluminium, gold, copper, magnesium, a silicon simple substance, and a single crystal of halide of rare earth elements.

### (Electrode)

The transducer which has a piezoelectric material relating to the present invention is produced in such a way that electrodes are formed on both surfaces or a single surface of a piezoelectric body film (layer) and the piezoelectric body film is subjected to a polarization treatment. The electrode is formed by the use of an electrode material containing gold (Au), platinum (Pt), silver (Ag), palladium (Pd), copper (Cu), nickel (nickel), or tin (Sn) as a main substance.

At the time of forming an electrode, first, underlying metals, such as titanium (Ti) and chromium (Cr), are formed with a thickness of 0.02 to 1.0 μm by a sputtering method. Subsequently, metals containing the above metal elements as main substance, and metal materials composed of alloys of them, and further insulating material partially if needed are made into a layer with a thickness of 1 to 10 μm by a sputtering method. In addition to the sputtering method, these electrode may be formed such that a conductive paste in which metal fine particles and a low melting point glass are mixed is made into a film by a screen printing, a dipping method, or a spraying method.

Thereafter, a predetermined voltage is supplied between the electrodes formed the both surfaces of the piezoelectric body film so as to polarize the piezoelectric body film, whereby a piezoelectric element is produced.

### (Ultrasound Probe)

The ultrasound probe relating to the present invention is employed as a probe for an ultrasonic medical image diagnostic apparatus provided with an ultrasonic transmitting transducer and an ultrasonic receiving transducer.

In the present invention, both of transmission and reception of ultrasonic waves may be conducted by one transducer. However, more preferably, transducers are separated for transmission and for reception and are constituted in a probe.

As a piezoelectric material which constitutes a transmitting transducer (a transducer for transmitting ultrasonic waves), a conventionally well-known ceramic inorganic piezoelectric material or an organic piezoelectric material may be employed.

In the ultrasound probe relating to the present invention, the ultrasonic receiving transducer of the present invention may be arranged on or in parallel to a transmitting transducer.

As a more desirable embodiment, preferable is a structure in which the ultrasonic receiving transducer of the present invention is laminated on an ultrasonic transmitting transducer. In this case, the ultrasonic receiving transducer of the present invention may be laminated on the ultrasonic transmitting transducer on the condition that the ultrasonic receiving transducer is pasted on other polymer material (the above polymer (resin) film with a relatively low specific permittivity, such as a polyester film as a support). The total film thickness of the receiving transducer and the other polymer material is preferably matched with a desirable received frequency band on a point of the design of a probe. From the viewpoints of a practical ultrasonic medical image diagnostic apparatus and a realistic frequency band for collecting organism information, the thickness is preferably 40 150 μm.

In addition, the probe may be provided with a backing layer, a sound matching layer, an acoustic lens, and the like. The probe may be structured with multiple transducers having piezoelectric materials which are arranged in two dimensions. Multiple probes arranged in two dimensions are structured as a scanner which scans sequentially with the multiple probes so as to form an image.

### (Ultrasonic Medical Image Diagnostic Apparatus)

The abovementioned ultrasound probe relating to the present invention can be used for various modes of ultrasonic diagnostic apparatus. For example, preferable is an ultrasonic medical image diagnostic apparatus equipped with an ultrasound probe (probe) in which arranged is a piezoelectric body transducer which transmits ultrasonic waves to an analyte, such as a patient and receives the ultrasonic waves reflected by the analyte as an echo signal. Further, the ultrasonic medical image diagnostic apparatus is preferably equipped with a transmission and reception circuit to supply electric signals to the ultrasound probe so as to generate ultrasonic waves and to receive echo signals received by each piezoelectric body transducer of the ultrasound probe, and a transmission reception control circuit which controls transmission and reception of the transmission and reception circuit.

Furthermore, the ultrasonic medical image diagnostic apparatus is preferably equipped with an image data converting circuit to convert the echo signals received by the transmission and reception circuit into ultrasonic image data of an analyte, a display control circuit to control a monitor to display the converted ultrasonic image data, and a control circuit to control the entire body of the ultrasonic medical image diagnostic apparatus.

In such an ultrasonic medical image diagnostic apparatus, the transmission reception control circuit, the image data converting circuit, and the display control circuit are connected to the control circuit, and the control circuit controls the actions of each of these circuits. Each of the piezoelectric body transducers of an ultrasound probe is applied with electric signals so as to transmit ultrasonic wave to a analyte, and the ultrasound probe receives reflected waves caused by the mismatching of acoustic impedance in the analyte.

The abovementioned transmission and reception circuit corresponds to “a means for generating electric signals”, and an image data converting circuit corresponds to an “image processing means”.

According to the above ultrasonic diagnostic apparatus, with the utilization of the features of the ultrasonic receiving transducer of the present invention which is excellent in piezoelectric properties and heat resistance properties and is suitable for high frequency and a wide band, it becomes possible to obtain ultrasonic images improved in image quality, reproducibility and stability as compared with conventional technology.

### EXAMPLE

Hereafter, although the present invention will be explained with reference to examples, the present invention is not limited to these examples.

### (Production and Evaluation of an Organic Piezoelectric Material)

### Example 1

A polyvinylidene fluoride copolymer powder (weight average molecular weight: 290,000) in which a mole fraction of vinylidene fluoride (hereafter, referred to as VDF) and trifluoro ethylene (hereafter, referred to as 3FE) was 75:25 was dissolved in a mixture solvent of ethyl methyl ketone (hereafter, referred to as MEK) and dimethylformamide (hereafter, referred to as DMF) mixed at 9:1, and the resultant solution was cast to form a layer on a glass plate. Successively, the solvent in the layer was dried at ° C., whereby a film (organic piezoelectric material) with a thickness of about 140 μm and a melting point 155° C. was obtained.

This film was stretched four times at a room temperature by an uniaxial-stretching machine with a load cell in which a load applied on a chuck can be measured. At the time point that the four time stretching was completed, the tension in the stretching axial direction was 2.2 N per a unit width (mm). While keeping the stretched length, the stretching machine was heated to conduct a heat treatment at 135° C. for one hour. Subsequently, while a distance between chucks was being controlled so as not to make the tension to become 0 (relaxation treatment), the film is cooled to a room temperature. After the heat treatment, the thus-obtained film had a film thickness of 43 μm. Then, both surfaces of the obtained film were coated with vapor deposition of gold/aluminum such that the surface resistance became 20Ω or less, whereby a sample with a surface electrode was obtained. Successively, this electrode was subjected to a polarization treatment by being applied with an alternating voltage of 0.1 Hz at a room temperature. In the polarization treatment, a voltage was raised gradually from a low voltage until an electric field between the electrodes became 100 MV/m eventually. The final amount of polarization calculated from an amount of remanent polarization in the case where a piezoelectric material was deemed as a capacitor, that is, from an amount of accumulating electric charge for a layer thickness, an electrode area and an applied electric field. As a result, Sample 1 of the present invention was obtained. The stretching temperature of a sample, the stretching magnification, the tension immediately after the stretching, a heat treatment temperature, a heat treatment time, the tension during the heat treatment, and the amount of relaxation at the time of cooling were summarized in Table 1.

As with Sample 1, a film (organic piezoelectric material) with a thickness of about 140 μm was stretched four times at a room temperature. At the time point that the four time stretching was completed, the tension in the stretching axial direction was 2.2 N per a unit width (mm). Subsequently, while the stretching machine was being heated to 135° C., and while the tension was being controlled to become 0.1 N/mm, the distance between the chucks in the stretching axial direction was shrunk. After the temperature in the stretching machine became 135° C., a heat treatment was conducted for one hour under the control of the tension, whereby Sample 2 was obtained. Hereafter, an amount of remanent polarization was calculated as with Sample 1.

With regard to other Samples of the present invention and Comparative samples, film formation and provision of electrodes were achieved in the same way as Sample on the conditions shown in Table, whereby Samples 3 to 8 to which a polarization treatment was finished were obtained.

### [Flatness of an Organic Piezoelectric Material]

The organic piezoelectric material with an electrode obtained in the above ways was cut out into a rectangle with a length of 100 min in a stretching direction and a length of 20 mm in a direction perpendicular to the stretching direction. The cut-down piezoelectric film was placed on a transparent acrylic board, and a load of 10 kg/cm² was pushed onto it from the upper side across a piece of metal plate, and then the flatness of the piezoelectric film was evaluated by the visual observation from the acrylic board side. With regard to Sample 8, the evaluation was conducted such that the cut-out direction was made perpendicular.

A: No wrinkle, and no crack on the electrode and the piezoelectric body film

B: No wrinkle, but cracks occurred on the electrode and the piezoelectric body film, so practically unemployable

C: Wrinkle occurred, and cracks occurred on the electrode and the piezoelectric body film, so practically unemployable

### [Evaluation Method of an Organic Piezoelectric Material]

Lead wires were attached to the electrodes of both surfaces of respective Samples with an electrode obtained in the above ways, and then the Samples were subjected to frequency sweep at 600 points with equal interval from 40 Hz to 110 MHz under the atmosphere of 25° C. by the use of an impedance analyzer 4294A manufactured by Agilent Technologies Corporation. The value of a specific permittivity at a thickness resonance frequency was obtained. Similarly, a peak frequency P of the resistance near the thickness resonance frequency and a peak frequency S of conductance were obtained respectively, and an electromechanical coupling factor kₜ was calculated by the following formula.

kₜ=(α/tan(α))^{1/2}, where α=(π/2)×(S/P)

A method of obtaining an electromechanical coupling factor from a thickness resonance frequency by the use of an impedance analyzer was pursuant to paragraph 42.6 in thickness longitudinal vibration of a disc-shaped transducer described in the electric test procedure of a piezoelectric ceramic transducer in Japan Electronics and Information Technology Industries Association Standard JETTA EM-4501 (former EMAS-6100).

The above-mentioned evaluation results are shown in Table 1.

TABLE 1

| | Melting | | | | | | | | | | | | |
|---|---|---|---|---|---|---|---|---|---|---|---|---|---|
| | point of an | | | | | | | | | | | | |
| | organic | Stretching | | | Releasing | Heat treatment | | | amount of | amount of | | | |
| Sam- | piezo- | Temper- | Magni- | Ten- | of tension | Temper- | | Ten- | relaxation | polari- | | Visual | |
| ple | electric | ature | fication | sion | after | ature | Time | sion | at the time | zation | Coupling | rank of | Re- |
| No. | material | ° C. | times | N/mm | stretching | ° C. | hr. | N/mm | of cooling*¹ % | mC/m² | constant | flatness | marks |
| 1 | 155 | 28 | 4.0 | 2.1 | No | 135 | 1.0 | 2.1 | +9 | 83 | 0.33 | A | Inv. |
| 2 | 155 | 28 | 4.0 | 2.1 | No | 135 | 1.0 | 0.1 | −10 | 85 | 0.34 | A | Inv. |
| 3 | 155 | 28 | 4.0 | 2.1 | Yes*² | 135 | 1.0 | No | No | 83 | 0.33 | C | Comp. |
| 4 | 155 | 28 | 6.0 | 2.2 | No | 135 | 1.0 | 2.2 | +9 | 80 | 0.32 | A | Inv. |
| 5 | 155 | 50 | 4.0 | 1.4 | No | 135 | 1.0 | 1.4 | +8 | 78 | 0.31 | A | Inv. |
| 6 | 155 | 28 | 4.0 | 2.1 | No | 135 | 0.3 | 2.1 | +9 | 60 | 0.24 | A | Inv. |
| 7 | 155 | 28 | 4.0 | 2.1 | No | 150 | 1.0 | 2.1 | Fracture | — | — | — | Comp. |
| 8 | 155 | 28 | 4.0 | 2.1 | No | 135 | 1.0 | 2.1 | +9 | 80 | 0.32 | B*³ | Inv. |

*¹An amount of relaxation (%) = (a travel distance of chuck after the completion of the heat treatment)/(a distance between chucks after the completion of the heat treatment) × 100
With regard to the sign of the travel distance, the when the travel direction is the same with the stretching direction, the sign is made minus, and when the travel direction is reverse to the stretching direction, the sign is made plus.
*²Clips are removed after the stretching, and then heat treatment was conducted anew.
*³The cut-out direction is different from others.
Inv.: Inventive, Comp.: Comparative

As can be understood from the results shown in Table 1, it turns out that the Samples prepared within the scope of the present invention are excellent in piezoelectric properties, and that since the Samples are excellent in flatness and piezoelectric properties, he Samples are excellent in processing adequacy for a transducer.

### Example 2

### (Production and Evaluation of a Probe)

### (Production of Piezoelectric Material for Transmission)

Component raw materials of caco₃, La₂O₃, Bi₂O₃ and TiO₂ and accessory component raw materials of MnO were prepared. The component raw materials were weighed such that the composition of the components became (Ca_{0.97}La_{0.03})Bi_{4.01}Ti₄O₁₅. Then, the component raw materials and the accessory component raw materials were added in pure water, mixed in the pure water by a ball mill in which media made from zirconia was put, and dried sufficiently, whereby mixed powder was obtained. The obtained powder was shaped in a temporary form and was preliminary fired in air at 800° C. for two hours, whereby a preliminary fired body was produced. Next, the obtained preliminary fired body was added in pure water, subjected to fine grinding in the pure water by a ball mill in which media made from zirconia was put, and dried, whereby raw material powder of a piezoelectric ceramic was prepared. In the fine grinding, time to conduct the fine grinding and the condition of the fine grinding were adjusted such that the raw material powder of a piezoelectric ceramic with a particle size of 100 nm was obtained. Into respective raw material powders of piezoelectric ceramics different in particle size, 6 mass % of pure water was added as a binder, and the raw material powders were subjected to a press shaping so as to become a plate-like temporarily-shaped body with a thickness of 100 μm. Then, this plate-like temporarily-shaped body was subjected to vacuum packaging, and shaped with a pressure of 235 MPa. Next, the above shaped body was calcined. As a result, a sintered body with a thickness of 20 μm was obtained. In the calcining, the calcining temperature was 1100° C. An electric field higher 1.5 times or more than a coercive electric field was applied for 1 minute so that the sintered body was subjected to polarization treatment.

### (Production of a Laminated Transducer for Reception)

The film (organic piezoelectric material) of a polyvinylidene fluoride copolymer which was produced in Example 1 and have been already irradiated with electron beams and a polyester with a thickness of 50 μm were pasted with epoxy adhesive, whereby a laminated transducer was produced.

Next, in accordance with the conventional method, the laminated transducer for reception was laminated on the above-mentioned piezoelectric material for transmission, and a backing layer and a sound matching layer were provided, whereby an ultrasound probe was made as a prototype.

As a comparative example, a probe was produced in the same way as the above ultrasound probe except that in place of the above laminated transducer for reception, a laminated transducer for reception which employed only a film (organic piezoelectric material) of a polyvinylidene fluoride copolymer was laminated on above-mentioned piezoelectric material for transmission.

Subsequently, the above-mentioned two kinds of ultrasound probes were evaluated by being subjected to measurement of reception sensitivity and eleetiical breakdown strength.

With regard to the reception sensitivity, a fundamental frequency f₁ with 5 MHz was transmitted, and a reception relative sensitivity was obtained for 10 MHz as a secondary harmonic f₂, 15 MHz as a third harmonic, and 20 MHz as a fourth harmonic. The reception relative sensitivity was measured by the use of a sound intensity measurement system Model 805 (1-50 MHz) manufactured by Sonora Medical System Corporation (Sonora Medical System Inc: 2021 Miller Drive Longmont, Colo. (0501 USA)).

In the measurement of electrical breakdown strength, the load power P was increased to five times and the test was conducted for 10 hours, thereafter the load power was returned to the basic power and the relative reception sensitivity was evaluated. When the lowering of the sensitivity was 1% or less of the sensitivity before the load test, the evaluation was “good”, when the lowering of the sensitivity exceeded 1% and was less than 10%, the evaluation was “acceptable”, and when the lowering of the sensitivity was more than 10%, the evaluation was “bad”.

In the above-mentioned evaluation, the probe provided with the transducer laminated with the piezoelectric (body) for reception relating to the present invention had relative reception sensitivity higher about 1.2 times than Comparative example, and it was confirmed that its electrical breakdown strength was good. That is, it was confirmed that the ultrasonic transducer of the present invention can be employed conveniently also for a probe used for an ultrasonic medical image diagnostic apparatus.

1-7. (canceled)

8. An organic piezoelectric material shaped in a film, comprising:
the organic piezoelectric material having a melting point,
wherein the organic piezoelectric material is produced by being subjected to a heat treatment at a temperature from a room temperature to a temperature lower by 10° C. than the melting point of the organic piezoelectric material while being applied with tension, and successively, by being subjected to a relaxation treatment while being cooled to a room temperature.

9. The organic piezoelectric material described in claim 8, wherein the organic piezoelectric material is subjected to biaxially stretching or uniaxially stretching, and after the stretching, without allowing stress applied on the organic piezoelectric material to become zero, the organic piezoelectric material is subjected to the heat treatment, and successively, to the relaxation treatment.

10. The organic piezoelectric material described in claim 8, wherein in the heat treatment, the organic piezoelectric material is heated at a temperature of 100° C. or more and 140° C. or less for 30 minutes or more and 10 hours or less while being applied with tension, and successively, in the relaxation treatment, while the organic piezoelectric material is being cooled to a room temperature, a length of the organic piezoelectric material is changed by −15% or more and +10% or less so as to relax stress applied on the organic piezoelectric material.

11. The organic piezoelectric material described in claim 8, wherein the organic piezoelectric material is composed of a copolymer of vinylidene fluoride and trifluoro ethylene, and the copolymer contains the vinylidene fluoride in an amount of 95 to 60 mol % and the trifluoro ethylene in an amount of 5-40 mol %.

12. The organic piezoelectric material described in claim 8, wherein the organic piezoelectric material has an electromechanical coupling factor of 0.3 or more.

13. An ultrasonic transducer, comprising
the organic piezoelectric material described in claim 8, wherein a direction in which the organic piezoelectric material is subjected to the relaxation treatment is parallel to a long side direction of the ultrasonic transducer.

14. An ultrasonic medical image diagnostic apparatus, comprising:
a generating section to generate electric signals;
an ultrasound probe including a plurality of transducers to transmit ultrasonic waves to an analyte in response to the electric signals and to generate reception signals corresponding to reflection waves received from the analyte; and
an image processing section to produce an image of the analyte corresponding to the reception signals generated by the ultrasound probe;
wherein the plurality of transducers include both of a transmission transducer for transmitting ultrasonic waves and a reception transducer for receiving ultrasonic waves, and at least one of the transmission transducer and the reception transducer is the ultrasonic transducer described in claim 13.

15. A method of producing an organic piezoelectric material shaped in a film, comprising:
a heat treatment process of heating the organic piezoelectric material at a temperature from a room temperature to a temperature lower by 10° C. than the melting point of the organic piezoelectric material while applying tension to the organic piezoelectric material; and successively,
a relaxation treatment process of relaxing stress applied on the organic piezoelectric material while the organic piezoelectric material is being cooled to a room temperature.

16. The producing method described in claim 15, further comprising:
a stretching process of stretching the organic piezoelectric material biaxially or uniaxially,
wherein after the stretching, without allowing stress applied on the organic piezoelectric material to become zero, the organic piezoelectric material is subjected to the heat treatment process, and successively, to the relaxation treatment process.

17. The producing method described in claim 15, wherein in the heat treatment process, the organic piezoelectric material is heated at a temperature of 100° C. or more and 140° C. or less for 30 minutes or more and 10 hours or less while being applied with tension, and successively, in the relaxation treatment process, while the organic piezoelectric material is being cooled to a room temperature, a length of the organic piezoelectric material is changed by −15% or more and +10% or less so as to relax stress applied on the organic piezoelectric material.
